(19) United States
(12) Patent Application Publication
(10) Pub. No.: US 2023/0200837 A1
(43) Pub. Date: Jun. 29, 2023

(54) TRAINING DATA GENERATION METHOD, CONTROL DEVICE, AND CONTROL METHOD

(71) Applicant: OLYMPUS MEDICAL SYSTEMS CORP., Tokyo (JP)

(72) Inventor: Gen KATO, Tokyo (JP)

(73) Assignee: OLYMPUS MEDICAL SYSTEMS CORP., Tokyo (JP)

(21) Appl. No.: 18/085,635

(22) Filed: Dec. 21, 2022

Related U.S. Application Data
(60) Provisional application No. 63/293,900, filed on Dec. 27, 2021.

(51) Int. Cl. A61B 17/32

(52) U.S. Cl. CPC A61B 17/320092; A61B 2017/00017

(57) ABSTRACT

A training data generation method includes: obtaining output information related to an electrical characteristic value in an energy treatment tool when ultrasound energy is being applied from the energy treatment tool to a body tissue; obtaining photography data that contains a photograph taken of a state in which the ultrasound energy is being applied to the body tissue; obtaining a label from the photography data; and adding the label to the output information to generate the training data.

### CROSS-REFERENCE TO RELATED APPLICATION

This application is based on and claims priority under 35 U.S.C. § 119 to U.S. Provisional Application No. 63/293,900, filed Dec. 27, 2021, the entire contents of which are incorporated herein by reference.

### BACKGROUND

### 1. Technical Field

The present disclosure relates to a training data generation method, a control device, and a control method.

### 2. Related Art

In the related art, a treatment system is known in which the body tissue is treated by applying a treatment energy thereto from an energy treatment tool (for example, refer to International Laid-open Pamphlet No. 2018/011918).

In the treatment system disclosed in Patent International Laid-open Pamphlet No. 2018/011918, the body tissue is treated by applying ultrasound vibration thereto. That is, in that treatment system, the ultrasound energy is used as the treatment energy.

### SUMMARY

In some embodiments, a training data generation method is implemented by a processor of a training data generation device. The training data generation method includes: obtaining output information related to an electrical characteristic value in an energy treatment tool when ultrasound energy is being applied from the energy treatment tool to a body tissue; obtaining photography data that contains a photograph taken of a state in which the ultrasound energy is being applied to the body tissue; obtaining a label from the photography data; adding the label to the output information to generate the training data.

In some embodiments, a control device comprising a processor, the processor being configured to: obtain output information related to an electrical characteristic value in an energy treatment tool when ultrasound energy is being applied from the energy treatment tool to a body tissue; input the output information to an estimation model generated as a result of performing machine learning; obtain relevant information related to treatment of the body tissue from the estimation model.

In some embodiments, a control method implemented by a processor of a control device, the control method comprising: obtaining output information related to an electrical characteristic value in an energy treatment tool when ultrasound energy is being applied from the energy treatment tool to a body tissue; inputting the output information to an estimation model generated as a result of performing machine learning; obtaining relevant information related to treatment of the body tissue from the estimation model.

### BRIEF DESCRIPTION OF THE DRAWINGS

FIG. 1 is a diagram illustrating a treatment system according to a first embodiment.

FIG. 2 is a diagram illustrating a transducer unit.

FIG. 3 is a block diagram illustrating a configuration of a control device.

FIG. 4 is a diagram illustrating a configuration of an estimation model generation system.

FIG. 5 is a flowchart for explaining a training data generation method.

FIG. 6 is a flowchart for explaining an estimation model generation method.

FIG. 7 is a flowchart for explaining a control method.

FIG. 8 is a diagram for explaining the effect of the first embodiment.

FIG. 9 is a diagram illustrating a configuration of an estimation model generation system according to a second embodiment.

FIG. 10 is a flowchart for explaining the training data generation method.

FIG. 11 is a flowchart for explaining the estimation model generation method.

FIG. 12 is a flowchart for explaining the control method.

FIG. 13 is a diagram for explaining a first modification example of the first embodiment.

FIG. 14 is a diagram for explaining a second modification example of the first and second embodiments.

FIG. 15 is a diagram for explaining a third modification example of the second embodiment.

### DETAILED DESCRIPTION

Illustrative embodiments (hereinafter, called embodiments) of the disclosure are described below with reference to the accompanying drawings. However, the disclosure is not limited by the embodiments described below. Moreover, in the explanation of the drawings, the same constituent elements are referred to by the same reference numerals.

### First Embodiment

Overall Configuration of Treatment System

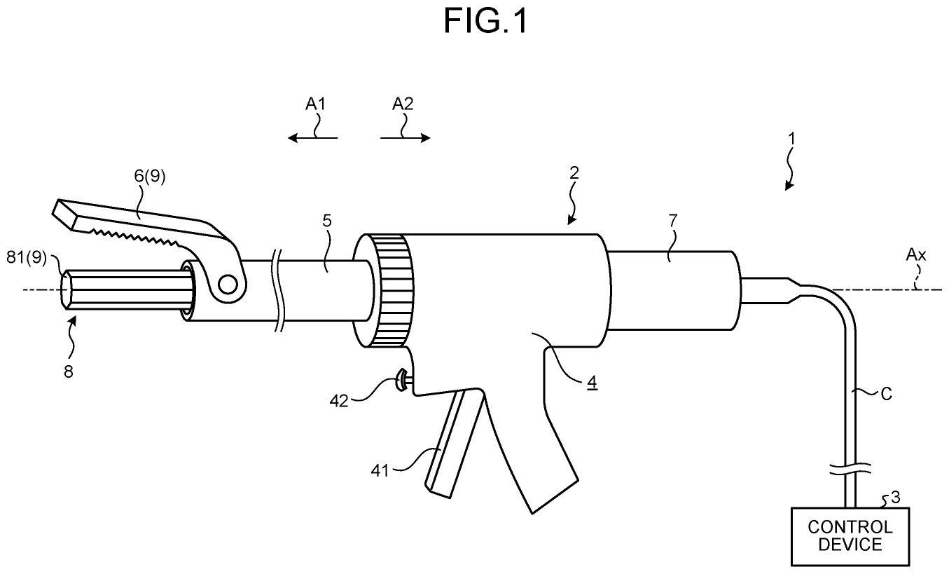

FIG. 1 is a diagram illustrating a treatment system 1 according to a first embodiment.

In the treatment system 1, a treatment energy is applied to that site of the body tissue which is to be treated (hereinafter, called the target site), so that the target site is treated. In the first embodiment, the ultrasound energy and the high-frequency energy is used as the treatment energy. Herein, the treatment implies coagulation (sealing) of the target site or incision of the target site. However, coagulation (sealing) of the target site and incision of the target site can be performed at the same time as part of the treatment. As illustrated in FIG. 1, the treatment system 1 includes an energy treatment tool 2 and a control device 3.

Configuration of Energy Treatment Tool

The energy treatment tool 2 is an ultrasound treatment tool including a Bolt-clamped Langevin Transducer (BLT). As illustrated in FIG. 1, the energy treatment tool 2 includes a handle 4, a sheath 5, a jaw 6, a transducer unit 7, and a vibration transmission member 8.

The handle 4 represents the portion held in a hand by the operator. As illustrated in FIG. 1, the handle 4 has an operation knob 41 and an operation button 42 disposed thereon.

The sheath 5 is cylindrical in shape. In the following explanation, the central axis of the sheath 5 is referred to as a central axis Ax (see FIG. 1). Moreover, in the following explanation, along the central axis Ax, one side is referred to as a front end side A1 (see FIG. 1), and the other side is referred to as the proximal end side A2 (see FIG. 1). The sheath 5 is attached to the handle 4, while some part of the sheath 5 on the proximal end side A2 is kept inserted inside the handle 4 from the front end side A1 of the handle 4.

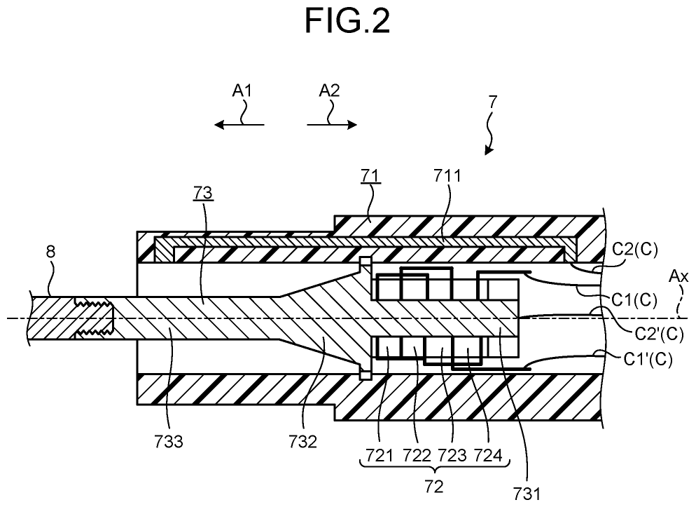

FIG. 2 is a diagram illustrating the transducer unit 7. More particularly, FIG. 2 is a cross-sectional view obtained when the transducer unit 7 is cut along the plane including the central axis Ax.

As illustrated in FIG. 2, the transducer unit 7 includes a transducer case 71, an ultrasound transducer 72, and a horn 73.

The transducer case 71 extends in a linear manner along the central axis Ax; and is attached to the handle 4, while some part of the transducer case 71 on the front end side A1 is kept inserted inside the handle 4 from the proximal end side A2 of the handle 4.

The ultrasound transducer 72 is housed inside the transducer case 71 and generates ultrasound vibration under the control of the control device 3. In the first embodiment, the ultrasound transducer 72 is an BLT that includes a plurality of piezoelectric elements 721 to 724 laminated along the central axis Ax. In the first embodiment, although there are four piezoelectric elements 721 to 724, the number of piezoelectric elements is not limited to four and it is possible to have some other count.

The horn 73 is housed inside the transducer case 71 and expands the amplitude of the ultrasound vibration generated by the ultrasound transducer 72. The horn 73 has an elongated shape extending in a linear manner along the central axis Ax. As illustrated in FIG. 2, the horn 73 is configured by arranging a first mounting portion 731, a cross-section variation portion 732, and a second mounting portion 733 in that order from the proximal end side A2 toward the front end side A1.

In the first mounting portion 731, the ultrasound transducer 72 is mounted.

In the cross-section variation portion 732, the cross-sectional area goes on decreasing toward the front end portion A1, so that the amplitude of the ultrasound vibration is expanded.

In the second mounting portion 733, the end portion on the proximal end side A2 of the vibration transmission member 8 is mounted.

The jaw 6 and the vibration transmission member 8 grasp the target site as well as give treatment to the target site by applying the ultrasound energy and the high-frequency energy to the target site.

More particularly, the jaw 6 is made of an electroconductive material such as a metal, and is rotatably attached to the end portion on the front end side A1 of the sheath 5. Then, the jaw 6 grasps the target site along with a treatment portion 81 (see FIG. 1) that constitutes the vibration transmission member 8.

Meanwhile, although not specifically illustrated in the drawings, inside the handle 4 and the sheath 5, an opening-closing mechanism is installed that, in response to the operation of the operation knob 41 by the operator, opens and closes the jaw 6 with respect to the treatment portion 81. Moreover, in the jaw 6, a resin pad (not illustrated) is attached to the surface that faces the treatment portion 81. On account of being electrically insulating, the pad has the function of preventing the occurrence of electrical short circuit between the jaw 6 and the vibration transmission member 8. Moreover, when the incision of the target site attributed to the ultrasound vibration is complete, the pad prevents the vibration transmission member 8, which is performing ultrasound vibration, from colliding with the jaw 6 and getting damaged.

The vibration transmission member 8 is made of an electroconductive material such as a metal, and has an elongated shape extending in a linear manner along the central axis Ax. As illustrated in FIG. 1, the vibration transmission member 8 is inserted inside the sheath 5, with the treatment portion 81 representing the end portion on the front end side A1 remaining protruded to the outside. Moreover, the end portion on the proximal end side A2 of the vibration transmission member 8 is connected to the second mounting portion 733 as illustrated in FIG. 2. Regarding the ultrasound vibration that is generated by the ultrasound transducer 72 and that has passed through the horn 73, the vibration transmission member 8 transmits the ultrasound vibration from the proximal end side A2 to the front end side A1, and applies the ultrasound vibration to the target site that is being grasped between the treatment portion 81 and the jaw 6. As a result, the target site gets treated. That is, the target site gets treated on account of being applied with the ultrasound energy from the treatment portion 81.

Configuration of Control Device

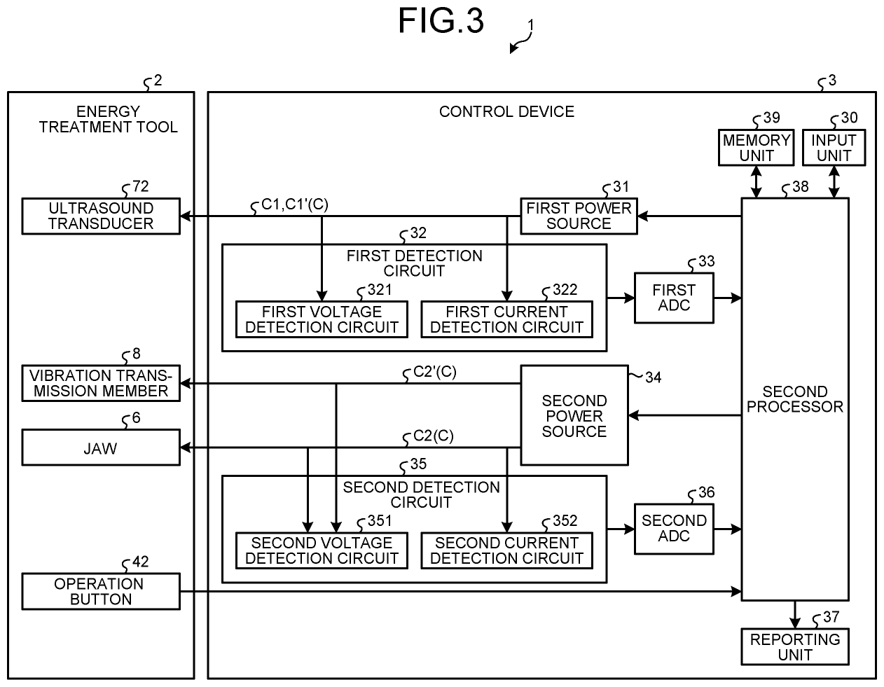

FIG. 3 is a block diagram illustrating a configuration of the control device 3.

The control device 3 is electrically connected to the energy treatment tool 2 by electric cables C (see FIG. 1), and comprehensively controls the operations of the energy treatment tool 2. As illustrated in FIG. 3, the control device 3 includes a first power source 31, a first detection circuit 32, a first ADC (Analog-to-Digital Converter) 33, a second power source 34, a second detection circuit 35, a second ADC 36, a reporting unit 37, a second processor 38, a memory unit 39, and an input unit 30.

Herein, a pair of transducer lead wires C1 and C1′ constituting the electric cables C is jointed to the ultrasound transducer 72 as illustrated in FIG. 2. Meanwhile, in FIG. 3, for the purpose of illustration, only a single pair of transducer lead wires C1 and C1′ is illustrated.

Under the control of the second processor 38, the first power source 31 outputs a first driving signal, which represents the electric power enabling generation of ultrasound vibration, to the ultrasound transducer 72 via the pair of transducer lead wires C1 and C1′. As a result, the ultrasound transducer 72 generates ultrasound vibration.

The first detection circuit 32 includes a first voltage detection circuit 321 representing a voltage sensor meant for detecting the voltage value, and includes a first current detection circuit 322 representing a current sensor meant for detecting the electric current value; and detects, over time, a US signal (an analog signal) that corresponds to the first driving signal being supplied to the ultrasound transducer 72. The US signal is equivalent to an “electrical characteristic value in the energy treatment tool”.

More particularly, examples of the US signal include: the electric current value in the first driving signal (hereinafter, referred to as a US current); the voltage value in the first driving signal (hereinafter, referred to as a US voltage); the electric power value in the first driving signal (hereinafter, referred to as a US power); the ultrasound impedance value calculated from the US current and the US voltage (hereinafter, referred to as a US impedance value); and the frequency of the US current or the frequency of the US voltage (hereinafter, referred to as a US frequency).

The first ADC 33 converts the US signal (an analog signal), which is output from the first detection circuit 32, into a digital signal. Then, the first ADC 33 outputs the post-conversion US signal (a digital signal) to the second processor 38.

As illustrated in FIG. 2, in the transducer case 71, a first conductive member 711 is disposed that extends from the end portion on the proximal end side A2 toward the end portion on the front end side A1. Moreover, in the sheath 5, although not illustrated in the drawings, a second conductive member is disposed that extends from the end portion on the proximal end side A2 toward the end portion on the front end side A1 and that electrically connects the first conductive member 711 and the jaw 6. Furthermore, at the end portion on the proximal end side A2 of the first conductive member 711, a high-frequency lead wire C2 is jointed that constitutes the electric cables C. Moreover, to the first mounting portion 731, a high-frequency lead wire C2′ is jointed that constitutes the electric cables C.

Under the control of the second processor 38, the second power source 34 outputs a second driving signal, which represents a high-frequency electric power, to the jaw 6 and the vibration transmission member 8 via the pair of high-frequency lead wires C2 and C2′, the first conductive member 711, the second conductive member, and the horn 73. As a result, a high-frequency electric current flows to the target site that is grasped between the jaw 6 and the treatment portion 81. That is, a high-frequency energy gets applied to the target site. As a result of the flow of the high-frequency electric current, Joule heat is produced using which a treatment is given to the target site.

As explained above, the jaw 6 and the treatment portion 81 are equivalent to a pair of electrodes. Moreover, the jaw 6 and the treatment portion 81 are equivalent to an end effector 9 (see FIG. 1).

The second detection circuit 35 includes a second voltage detection circuit 351 representing a voltage sensor for detecting the voltage value, and includes a second current detection circuit 352 representing a current sensor for detecting the electric current value; and detects, over time, an HF signal (an analog signal) that corresponds to the second driving signal. The HF signal is equivalent to an “electrical characteristic value in the energy treatment tool”.

More particularly, examples of the HF signal include: the electric current value in the second driving signal (hereinafter, referred to as an HF current); the voltage value in the second driving signal (hereinafter, referred to as an HF voltage); the electric power value in the second driving signal (hereinafter, referred to as an HF power); the phase difference between the HF current and the HF voltage (hereinafter, referred to as an HF phase difference); and the impedance value of the target site calculated from the HF current and the HF voltage (hereinafter, referred to as an HF impedance value).

The second ADC 36 converts the HF signal (an analog signal), which is output from the second detection circuit 35, into a digital signal. Then, the second ADC 36 outputs the post-conversion HF signal (a digital signal) to the second processor 38.

The reporting unit 37 reports predetermined information under the control of the second processor 38. Examples of the reporting unit 37 include: an LED that reports predetermined information by illumination, or by flashing, or according to the color at the time of illumination; a display device that displays predetermined information; and a speaker that outputs predetermined information in the form of sounds. As far as the position of the reporting unit 37 is concerned, it can be installed either in the control device 3 as illustrated in FIG. 3 or in the energy treatment tool 2.

The second processor 38 is configured using a controller such as a CPU (Central Processing Unit) or an MPU (Micro Processing Unit), or using an integrated circuit such as an ASIC (Application Specific Integrated Circuit) or an FPGA (Field Programmable Gate Array); and controls the operations of the entire treatment system 1.

Regarding the detailed functions of the second processor 38, the explanation is given later in a section called “control method”.

The memory unit 39 is used to store various programs (including a control program) that are executed by the second processor 38, and to store the information used for the operations performed by the second processor 38.

Examples of the information used for the operations performed by the second processor 38 include the setting value of the first driving signal, the setting value of the second driving signal, and an estimation model.

The estimation model is generated by an estimation model generation system 10 (explained later). Regarding the details of the estimation model and the estimation model generation system 10, the explanation is given later in sections called “configuration of estimation model generation system”, “training data generation method”, and “estimation model generation method”.

The input unit 30 is configured using a keyboard, a mouse, a switch, or a touch-sensitive panel, and receives user operations performed by the operator. Examples of a user operation include an input operation for inputting the setting values of the first driving signal and the second driving signal. Then, the input unit 30 outputs, to the second processor 38, an operation signal corresponding to the user operation.

Configuration of Estimation Model Generation System

Given below is the explanation about a configuration of the estimation model generation system 10.

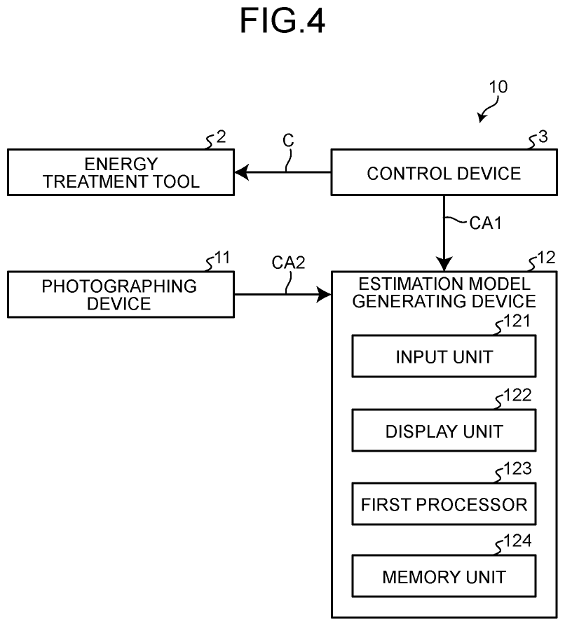

FIG. 4 is a diagram illustrating a configuration of the estimation model generation system 10.

The estimation model generation system 10 is a system for generating an estimation model by performing machine learning such as deep learning with the use of training data. As illustrated in FIG. 4, the estimation model generation system 10 includes the energy treatment tool 2, the control device 3, a photographing device 11, and an estimation model generating device 12.

The photographing device 11 is a camera that includes an imaging element such as a CCD (Charge Coupled Device) or a CMOS (Complementary Metal Oxide Semiconductor) for receiving the incident light and converting it into electrical signals, and that takes photographs of a specific region and generates photographed images. The photographing device 11 is communicably connected to the estimation model generating device 12 by a second transmission cable CA2. Thus, the photographing device 11 outputs the data of photographed images (equivalent to photography data) to the estimation model generating device 12 via the second transmission cable CA2.

Meanwhile, although the photographing device 11 is communicably connected to the estimation model generating device 12 by the second transmission cable CA2, that is not the only possible case. Alternatively, the photographing device 11 can be communicably connected to the estimation model generating device 12 in a wireless manner.

As illustrated in FIG. 4, the estimation model generating device 12 is communicably connected to the control device 3 and the photographing device 11 by a first transmission cable CA1 and the second transmission cable CA2, respectively, and comprehensively controls the operations performed by the entire estimation model generation system 10. The estimation model generating device 12 generates training data, as well as generates an estimation model by performing machine learning with the use of the training data. Meanwhile, although the estimation model generating device 12 is communicably connected to the control device 3 by the first transmission cable CA1, that is not the only possible case. Alternatively, the estimation model generating device 12 can be communicably connected to the control device 3 in a wireless manner.

As illustrated in FIG. 4, the estimation model generating device 12 includes an input unit 121, a display unit 122, a first processor 123, and a memory unit 124.

The input unit 121 is configured using a keyboard, a mouse, switches, or a touch-sensitive panel; and receives user operations. The input unit 121 outputs, to the first processor 123, an operation signal corresponding to a user operation.

The display unit 122 is a display configured using liquid crystals or organic EL (Electro Luminescence) and, under the control of the first processor 123, displays images based on video signals received from the first processor 123.

The first processor 123 is configured using a controller such as a CPU or an MPU, or using an integrated circuit such as an ASIC or an FPGA; and controls the operations of the entire estimation model generation system 10.

Regarding the detailed functions of the first processor 123, the explanation is given later in the sections of “training data generation method” and “estimation model generation method”.

The memory unit 124 is used to store various programs (including a training data generation program meant for generating training data, and an estimation model generation program meant for generating an estimation model), and to store the information used for the operations performed by the first processor 123.

Examples of the information used for the operations performed by the first processor 123 include a treatment completion detection model.

Regarding the details of the treatment completion detection model, the explanation is given later in the section of “training data generation method”.

Training Data Generation Method

Given below is the explanation of the training data generation method implemented by the first processor 123.

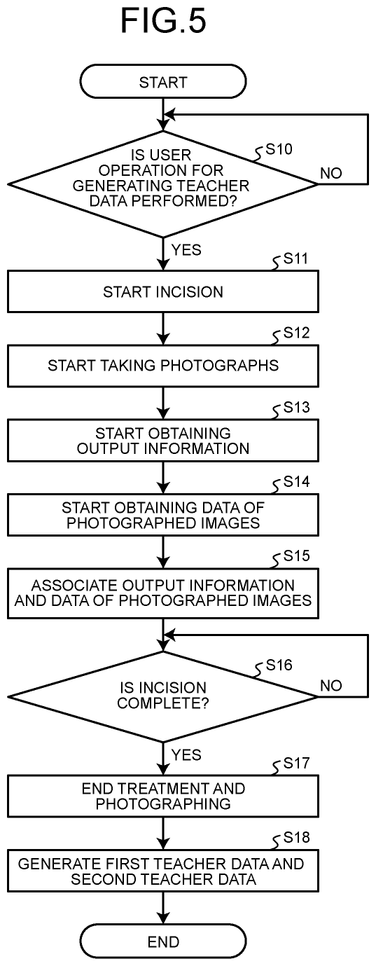

FIG. 5 is a flowchart for explaining the training data generation method.

In the following explanation, it is assumed that a specific target site is already grasped between the jaw 6 and the treatment portion 81.

Firstly, the first processor 123 constantly monitors whether or not a user operation for generating training data is performed by the user using the input unit 121 (Step S10). Until it is determined that a user operation for generating training data is performed, the first processor 123 repeatedly performs the determination at Step S10.

When it is determined that a user operation for generating training data is performed (Yes at Step S10), the first processor 123 outputs a control signal to the second processor 38 via the first transmission cable CA1. According to the control signal, the second processor 38 controls the operations of the first power source 31 and the second power source 34, and applies an ultrasound energy and a high-frequency energy to the target site that is grasped between the jaw 6 and the treatment portion 81. That is, the first processor 123 starts the treatment of the target site (Step S11). Then, the second processor 38 outputs output information (1) to output information (11) explained below to the estimation model generating device 12 via the first transmission cable CA1.

The output information (1) represents the elapsed time since the start of application of the ultrasound energy and the high-frequency energy to the target site.

The output information (2) represents the US current, from among the US signals detected by the first detection circuit 32, while the ultrasound energy and the high-frequency energy is being applied to the target site.

The output information (3) represents the US voltage, from among the US signals detected by the first detection circuit 32, while the ultrasound energy and the high-frequency energy is being applied to the target site.

The output information (4) represents the US power, from among the US signals detected by the first detection circuit 32, while the ultrasound energy and the high-frequency energy is being applied to the target site.

The output information (5) represents the US impedance value, from among the US signals detected by the first detection circuit 32, while the ultrasound energy and the high-frequency energy is being applied to the target site.

The output information (6) represents the US frequency, from among the US signals detected by the first detection circuit 32, while the ultrasound energy and the high-frequency energy is being applied to the target site.

The output information (7) represents the HF current, from among the HF signals detected by the second detection circuit 35, while the ultrasound energy and the high-frequency energy is being applied to the target site.

The output information (8) represents the HF voltage, from among the HF signals detected by the second detection circuit 35, while the ultrasound energy and the high-frequency energy is being applied to the target site.

The output information (9) represents the HF power, from among the HF signals detected by the second detection circuit 35, while the ultrasound energy and the high-frequency energy is being applied to the target site.

The output information (10) represents the HF phase difference, from among the HF signals detected by the second detection circuit 35, while the ultrasound energy and the high-frequency energy is being applied to the target site.

The output information (11) represents the HF impedance value, from among the HF signals detected by the second detection circuit 35, while the ultrasound energy and the high-frequency energy is being applied to the target site.

The output information (1) to the output information (11) represents the information that enables estimation of the status of the treatment given to the target site.

Moreover, the first processor 123 outputs a control signal to the photographing device 11 via the second transmission cable CA2. According to the control signal, the photographing device 11 takes photographs of the status of the treatment given to the target site that is grasped between the jaw 6 and the treatment portion 81. That is, the first processor 123 starts taking photographs of the status of the treatment given to the target site (Step S12). Then, the photographing device 11 sequentially outputs the data of photographed images to the estimation model generating device 12 via the second transmission cable CA2.

Meanwhile, in FIG. 5, for the purpose of illustration, the operation at Step S12 is performed after the operation at Step S11. However, in practice, the operations at Steps S11 and S12 are performed in a substantially simultaneous manner.

After the operations at Steps S11 and S12 are performed, the first processor 123 starts obtaining the output information (1) to the output information (11), which is sequentially output from the control device 3, via the first transmission cable CA1 (Step S13). The output information (1) to the output information (11) is then subjected to necessary preprocessing in the estimation model generating device 12.

Moreover, the first processor 123 starts obtaining the data of photographed images, which is sequentially output from the photographing device 11, via the second transmission cable CA2 (Step S14).

Meanwhile, in FIG. 5, for the purpose of illustration, the operation at Step S14 is performed after the operation at Step S13. However, in practice, the operations at Steps S13 and S14 are performed in a substantially simultaneous manner.

After the operations at Steps S13 and S14 are performed, the first processor 123 sequentially stores, in the memory unit 124, the output information (1) to the output information (11) in a corresponding manner to the data of photographed images obtained at respectively substantially identical timings (Step S15).

After the operation at Step S15 is performed, the first processor 123 performs image recognition using the treatment completion detection model stored in the memory unit 124, and constantly monitors whether or not the incision of the target site, which is captured as the photographic subject in the photographed images stored in the memory unit 124, is complete (Step S16). Until it is determined that the incision of the target site is complete, the first processor 123 repeatedly performs the operation at Step S16.

The treatment completion detection model is generated as a result of performing machine learning using such training data in which information indicating whether or not the target site has been incised is assigned (labeled) to the photographed images capturing the jaw 6, the treatment portion 81, and the target site.

The treatment completion detection model is made of a neural network in which each layer includes one or more nodes. Meanwhile, there is no particular restriction on the type of machine learning. For example, as long as training data is prepared in which information indicating whether or not the target site has been incised is assigned (labeled) to a plurality of photographed images, and as long as the training data is input for learning in a calculation model that is based on a multilayered neural network; it serves the purpose. As far as the method for machine learning is concerned, for example, a method based on a DNN (deep neural network) of a multilayered neural network, such as a CNN (Convolutional Neural Network), can be implemented. Alternatively, as far as the method for machine learning is concerned, a method based on a recurrent neural network (RNN) can be implemented, or a method based on an LSTM (Long Short-Term Memory Units), which is obtained by expanding an RNN, can be implemented.

When it is determined that the incision of the target site is complete (Yes at Step S16); after the elapse of a predetermined period of time since that determination, via the second transmission cables CA1 and CA2, the first processor 123 instructs the control device 3 to end the treatment given to the target site and instructs the photographing device 11 to end the photographing (Step S17).

After the operation at Step S17 is performed, the first processor 123 adds the information obtained from the data of photographed images to the output information (1) to the output information (11) stored in the memory unit 124; and generates first training data and second training data explained below (Step S18).

More particularly, the first processor 123 generates the first training data in which, from among the output information (1) to the output information (11) stored in the memory unit 124, with respect to the output information (1) to the output information (11) obtained before the determination performed at Step S16 about the completion of incision, information indicating that the incision is not yet complete is added (labeled). The information indicating that the incision is not yet complete is equivalent to the information obtained from the abovementioned data of photographed images. Moreover, the first processor 123 generates the second training data in which, from among the output information (1) to the output information (11) stored in the memory unit 124, with respect to the output information (1) to the output information (11) obtained after the determination performed at Step S16 about the completion of incision, information indicating that the incision is complete is added (labeled). The information indicating that the incision is complete is equivalent to the information obtained from the abovementioned data of photographed images. Meanwhile, the first training data and the second training data does not contain the data of photographed images.

Then, the first processor 123 stores the first training data and the second training data in the memory unit 124.

In the training data generation method explained above, the first processor 123 generates the first training data and the second training data. However, that is not the only possible case. Alternatively, for example, the first training data and the second training data can be manually generated by performing the following processes from Process (1) to Process (4) in that order.

Process (1): the operator operates the photographing device 11 and starts taking photographs. The data of photographed images gets sequentially stored in an internal recording unit of the photographing device 11.

Process (2): the operator operates the input unit 30, applies the ultrasound energy and the high-frequency energy to the target site that is grasped between the jaw 6 and the treatment portion 81, and starts giving treatment to the target site. Herein, at the point of time of starting the treatment to the target site, an LED (Light Emitting Diode) installed in the energy treatment tool 2 is activated or a speaker installed in the energy treatment tool 2 or the control device 3 is made to output a sound, so that it becomes possible to know the point of time of starting the treatment to the target site among the data of photographed images obtained by the photographing device 11. Then, the second processor 38 sequentially outputs the output information (1) to the output information (11) to the estimation model generating device 12 via the first transmission cable CA1. In the estimation model generating device 12, the output information (1) to the output information (11) is subjected to necessary preprocessing and is converted into the format that is usable as the training data.

Process (3): once the treatment given to the target site is complete, the operator operates the photographing device 11 and ends the photographing, as well as operates the input unit 30 and stops the energy application.

Process (4): the operator performs a sorting operation so as to sort the post-preprocessing output information (1) to the post-preprocessing output information (11) into data obtained before the completion of the incision and data obtained after the completion of the incision. For example, the operator confirms the data of photographed images, which is recorded in the photographing device 11, on the display screen, and gets to know the number of seconds taken for completing the incision since the start of the treatment. Accordingly, the operator performs the sorting operation and generates the first training data in which information indicating that the incision is not yet complete is added (labeled) to the output information (1) to the output information (11) sorted as the data obtained before the completion of the incision. Moreover, the operator generates the second training data in which information indicating that the incision is complete is added (labeled) to the output information (1) to the output information (11) sorted as the data obtained after the completion of the incision.

Estimation Model Generation Method

Given below is the explanation of the estimation model generation method implemented by the first processor 123.

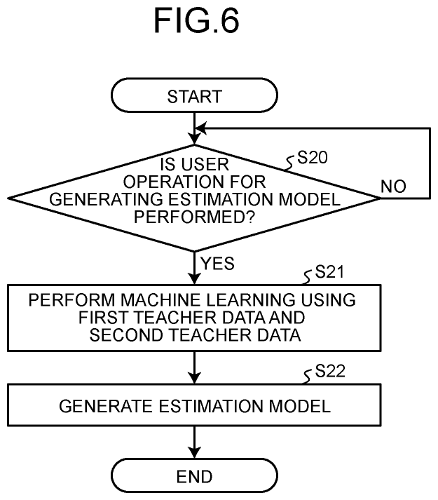

FIG. 6 is a flowchart for explaining the estimation model generation method.

Firstly, the first processor 123 constantly monitors whether or not a user operation for generating an estimation model is performed by the user using the input unit 121 (Step S20). Until it is determined that a user operation for generating an estimation model is performed, the first processor 123 repeatedly performs the determination at Step S20.

When it is determined that a user operation for generating an estimation model is performed (Yes at Step S20), the first processor 123 performs machine learning using the first training data and the second training data stored in the memory unit 124 (Step S21), and generates an estimation model meant for estimating relevant information that is related to the treatment given to the target site (Step S22). In the first embodiment, the relevant information indicates whether or not the incision of the target site is complete.

The estimation model is made of a neural network in which each layer includes one or more nodes. Meanwhile, there is no particular restriction on the type of machine learning. Thus, as long as a plurality of sets of first training data and second training data generated according to the training data generation method is input for learning in a calculation model that is based on a multilayered neural network; it serves the purpose. As far as the method for machine learning is concerned, for example, a method based on a DNN of a multilayered neural network, such as a CNN (Convolutional Neural Network), can be implemented. Alternatively, as far as the method for machine learning is concerned, a method based on a recurrent neural network (RNN) can be implemented, or a method based on an LSTM, which is obtained by expanding an RNN, can be implemented.

Then, the first processor 123 outputs the generated estimation model to the control device 3 via the first transmission cable CA1. Moreover, the control device 3 stores the estimation model in the memory unit 39.

Control Method

Given below is the explanation of the control method implemented by the second processor 38.

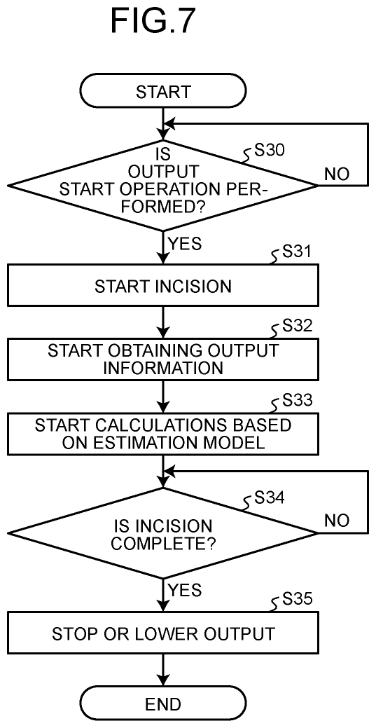

FIG. 7 is a flowchart for explaining the control method.

The present control method is implemented during an actual surgery. Hence, at the time of implementing the control method, only the treatment system 1 used; while the photographing device 11 and the estimation model generating device 12 are not used. Moreover, in the following explanation, it is assumed that a specific target site is already grasped between the jaw 6 and the treatment portion 81.

Firstly, the second processor 38 constantly monitors whether or not the operation button 42 is pressed (whether or not an output start operation is performed) by the operator (Step S30). Until it is determined that an output start operation is performed, the second processor 38 repeatedly performs the operation at Step S30.

When it is determined that an output start operation is performed (Yes at Step S30), the second processor 38 controls the operations of the first power source 31 and the second power source 34 and causes them to output the first driving signal and the second driving signal, respectively, corresponding to the setting values stored in the memory unit 39. As a result, the treatment energy (the ultrasound energy and the high-frequency energy) corresponding to the setting values of the first control signal and the second control signal gets applied to the target site that is grasped between the jaw 6 and the treatment portion 81. That is, the incision of the target site starts (Step S31).

After the operation at Step S31 is performed, the second processor 38 controls the operations of the first detection circuit 32 and the second detection circuit 35, and starts obtaining the output information (1) to the output information (11) (Step S32).

After the operation at Step S32 is performed, the second processor 38 starts calculations based on the estimation model stored in the memory unit 39 (Step S33).

More particularly, at Step S33, the second processor 38 treats the output information (1) to the output information (11) as the input data and performs calculations based on the estimation model; and outputs (estimates) the relevant information related to the treatment given to the target site as output data. In the first embodiment, as explained earlier, the relevant information indicates whether or not the incision of the target site is complete.

After the operation at Step S33 is performed, the second processor 38 refers to the calculation result obtained at Step S33 and constantly monitors whether or not the incision of the target site is complete (Step S34). Until it is determined that the incision of the target site is complete, the second processor 38 repeatedly performs the operation at Step S34.

More particularly, at Step S33, when the information indicating that the incision of the target site is complete is output as the output data, the second processor 38 determines that the incision of the target site is complete (Yes at Step S34). On the other hand, at Step S33, when the information indicating that the incision of the target site is not yet complete is output as the output data, the second processor 38 determines that the incision of the target site is not yet complete (No at Step S34).

When it is determined that the incision of the target site is complete (Yes at Step S34), the second processor 38 stops the operations of the first power source 31 and the second power source 34, and ends the incision of the target site (Step S35).

Meanwhile, at Step S35, although the operations of the first power source 31 and the second power source 34 are stopped, that is not the only possible case. Alternatively, the output of the ultrasound energy and the high-frequency energy can be lowered.

According to the first embodiment described above, it becomes possible to achieve the following effects.

In the training data generation method implemented by the first processor 123 according to the first embodiment, the information obtained from the data of photographed images taken by the photographing device 11 (i.e., the information indicating that the incision of the target site is not complete, or the information indicating that the incision of the target site is complete) is assigned (labeled) to the output information (1) to the output information (11), and the first training data and the second training data is generated. Moreover, in the estimation model generation method implemented by the first processor 123, machine learning is performed using the first training data and the second training data, and an estimation model is generated. Then, in the control method implemented by the second processor 38, calculations are performed based on the estimation model and it is estimated whether or not the incision of the target site is complete. If it is determined that the incision of the target site is complete, then the output of the ultrasound energy and the high-frequency energy is stopped. Thus, the application of the ultrasound energy and the high-frequency energy is not continued after the completion of the incision of the target site. As a result, no unnecessary damage is caused to the target site or the end effector 9.

Thus, the training data generation method, the control device 3, and the control method according to the first embodiment enable giving appropriate treatment to the target site.

Meanwhile, the state of the treatment given using the energy treatment tool 2 is impacted by the target tissues (whether or not a blood vessel is the target) and by the environment (high water content (in the blood) or low water content (less adherence of blood)). Hence, as far as estimating the completion of the incision of the tissues is concerned or estimating the temperature of the end effector 9 is concerned, the estimation accuracy has a limitation if only a single parameter (such as the US impedance value or the HF impedance value) is used. On the other hand, if a plurality of parameters is used while attempting to estimate the completion of the incision of the tissues or to estimate the temperature of the end effector, then it becomes necessary to implement a complex technique.

For example, in the case of applying only the ultrasound energy to the target site, the US impedance value undergoes changes due to the metamorphosis of the tissues or due to the fact that the pad provided on the surface of the jaw 6 facing the treatment portion 81 abuts against the treatment portion 81 after the completion of the incision of the target site. Moreover, the US frequency is impacted by the temperature of the vibration transmission member 8.

Meanwhile, for example, in the case of applying the ultrasound energy and the high-frequency energy to the target site in a simultaneous manner, only a high-frequency parameter (the HF impedance value or the HF phase difference) can also be used in detecting the completion of the incision of the target site or in estimating the temperature of the end effector 9. At the same time, there are also times when the accuracy of estimating the completion of the incision of the target site or the accuracy of estimating the temperature of the end effector 9 undergoes a decline due to the impact of the water content. However, if the ultrasound parameters and the high-frequency parameters are used in combination, the detection of the completion of the incision of the target site and the estimation of the temperature of the end effector 9 can be performed with a higher degree of accuracy as compared to the case of using only the parameters of only one type (either only the ultrasound parameters or only the high-frequency parameters).

If the number of parameters is increased, although the estimation accuracy can be expected to improve, the setting of the estimation method becomes more complex.

In that regard, if machine learning is utilized, the advantage is that the data under a variety of conditions (the environment, the target tissues, and the settings of the control device 3) can be learnt as the training data and an appropriate model can be created. That enables performing the estimation with accuracy using a plurality of parameters.

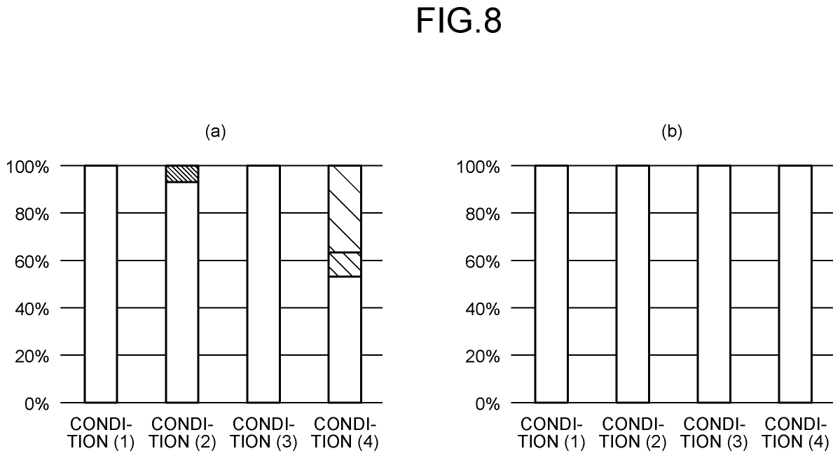

FIG. 8 is a diagram for explaining the effect of the first embodiment. More particularly, in (a) in FIG. 8 is illustrated the result of estimating the completion of the incision of the target site using only an ultrasound parameter (the US impedance value). In (b) in FIG. 8 is illustrated the result of estimating the completion of the incision of the target site when the control method according to the first embodiment is implemented. In FIG. 8, the open portions indicate the ratio of appropriate estimation of the completion of the incision. Moreover, the hatched portions indicate the ratio of misdetection in the case in which the incision of the target site is not yet complete in spite of the fact that the incision is estimated to have been already completed or in the case in which the completion of the incision is not yet estimated in spite of the fact that the incision of the target site is already complete. Furthermore, in FIG. 8, a condition (1) indicates that the incision is performed on the tissues having elasticity. A condition (2) indicates that the incision is performed on thin tissues. A condition (3) indicates that the incision is performed on soft tissues. A condition (4) indicates that the incision is performed on hard tissues.

If the completion of the incision of the target site is estimated using only an ultrasound parameter; then, as illustrated in (a) in FIG. 8, misdetection occurs under the conditions (2) and (4).

On the other hand, when the completion of the incision of the target site is estimated using the control method according to the first embodiment; as illustrated in (b) in FIG. 8, the estimation of the completion of the incision is appropriately estimated under all of the conditions (1) to (4).

### Second Embodiment

Given below is the description of a second embodiment.

In the following explanation, the configuration identical to the first embodiment is referred to by the same reference numerals, and the detailed explanation either is not given again or is given in a simplified manner.

In the second embodiment, the configuration of the estimation model generation system 10 is different than the configuration according to the first embodiment. In the following explanation, the estimation model generation system 10 and the photographing device 11 are referred to as an estimation model generation system 10A and a photographing device 11A, respectively.

Configuration of Estimation Model Generation System

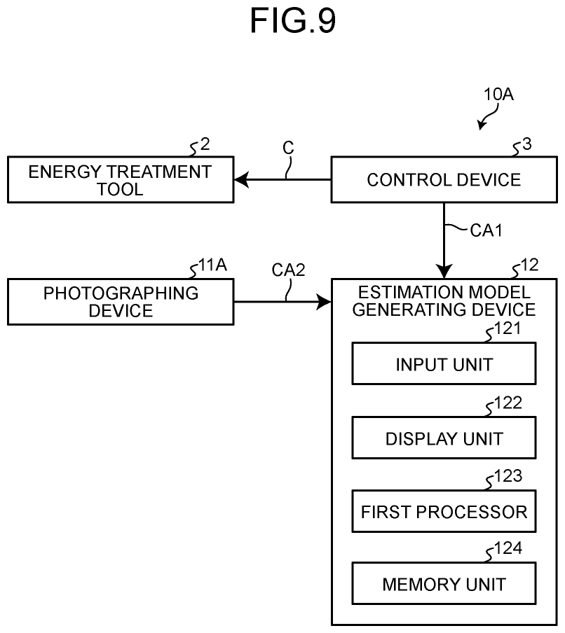

FIG. 9 is a diagram illustrating a configuration of the estimation model generation system 10A according to the second embodiment.

In the estimation model generation system 10A according to the second embodiment, as illustrated in FIG. 9, as compared to the estimation model generation system 10 according to the first embodiment, the photographing device 11A is different than the photographing device 11.

The photographing device 11A is a thermography that generates photographed images including temperature information indicating the temperature of the photographic subject. Then, the photographing device 11A outputs the data of photographed images (equivalent to photography data) to the estimation model generating device 12 via the second transmission cable CA2.

Training Data Generation Method

Given below is the explanation of the training data generation method implemented by the first processor 123.

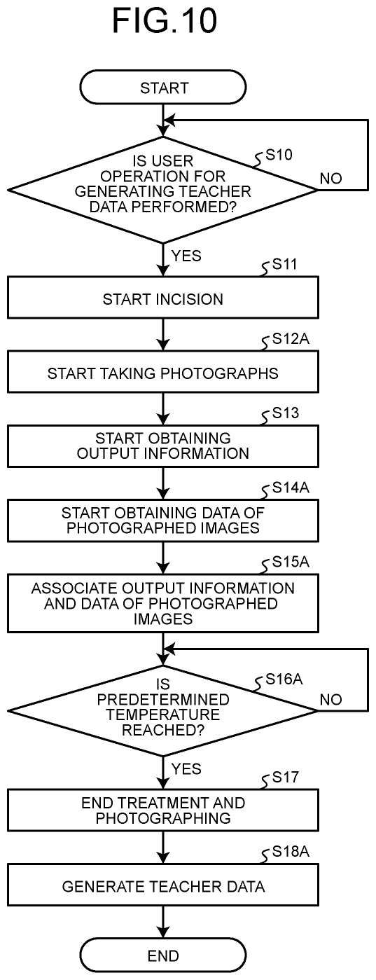

FIG. 10 is a flowchart for explaining the training data generation method.

In the training data generation method according to the second embodiment, as illustrated in FIG. 10, with reference to the training data generation method according to the first embodiment (see FIG. 5); Step S12, Steps S14 to S16, and Step S18 are replaced with Step S12A, Steps S14A to S16A, and Step S18A, respectively. Hence, the following explanation is mainly given about the operations performed at Step S12A, Steps S14A to S16A, and Step S18A.

The operation at Step S12A is performed in a substantially simultaneous manner to the operation performed at Step S11.

More particularly, at Step S12A, the first processor 123 according to the second embodiment outputs the control signal to the photographing device 11A via the second transmission cable CA2. According to the control signal, the photographing device 11A takes photographs of the status of the treatment given to the target site that is grasped between the jaw 6 and the treatment portion 81. That is, the first processor 123 starts taking photographs of the status of the treatment given to the target site. Then, the photographing device 11A sequentially outputs, to the estimation model generating device 12 via the second transmission cable CA2, the data of photographed images that includes temperature information indicating the temperature of at least either the end effector 9 representing the photographing subject or the target site representing the photographing subject.

The operation at Step S14A is performed in a substantially simultaneous manner to the operation at Step S13.

More particularly, at Step S14A, the first processor 123 according to the second embodiment starts sequentially obtaining the data of photographed images from the photographing device 11A via the second transmission cable CA2.

The operation at Step S15A is performed after the operations performed at Steps S13 and S14A.

More particularly, at Step S15A, the first processor 123 according to the second embodiment sequentially stores, in the memory unit 124, the output information (1) to the output information (11) in a corresponding manner to the data of photographed images obtained at respectively substantially identical timings.

The operation at Step S16A is performed after the operation performed at Step S15A.

More particularly, at Step S16A, the first processor 123 according to the second embodiment recognizes the temperature of at least either the end effector 9 or the target site based on the temperature information included in the obtained data of photographed images, and constantly monitors whether or not the recognized temperature has reached a predetermined temperature. Examples of the predetermined temperature include the temperature that affects the resistance property of the pad provided on the surface of the jaw 6 facing the treatment portion 81, and the temperature that is likely to cause excessive thermal invasion into the surrounding tissues. Until it is determined that the temperature of at least either the end effector 9 or the target site has reached the predetermined temperature, the first processor 123 repeatedly performs the operation at Step S16A. When it is determined that the temperature has reached the predetermined temperature (Yes at Step S16A), the system control proceeds to Step S17.

The operation at Step S18A is performed after the operation performed at Step S17.

More particularly, at Step S18A, the first processor 123 generates training data, which is explained below, by adding information obtained from the data of photographed images to the output information (1) to the output information (11) stored in the memory unit 124.

That is, the first processor 123 generates training data in which temperature information, which indicates the temperature of at least either the end effector 9 or the target site as specified in the data of photographed images associated to the output information (1) to the output information (11), is added (labeled) to the output information (1) to the output information (11) stored in the memory unit 124. Meanwhile, of the data of photographed images, only the temperature information is included in the training data, and the other data is not included.

Then, the first processor 123 stores the generated training data in the memory unit 124.

Estimation Model Generation Method

Given below is the explanation of the estimation model generation method implemented by the first processor 123.

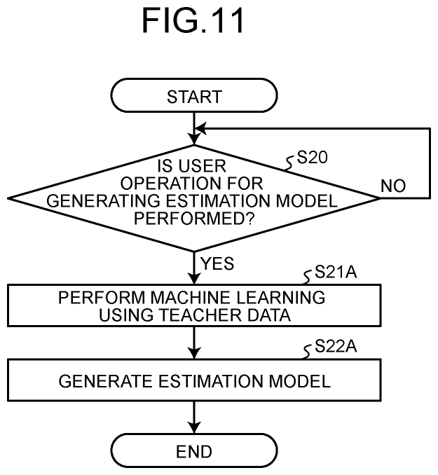

FIG. 11 is a flowchart for explaining the estimation model generation method.

In the estimation model generation method according to the second embodiment, as illustrated in FIG. 11, with reference to the estimation model generation method according to the first embodiment (see FIG. 6), Steps S21 and S22 are replaced with Steps S21A and S22A, respectively. Hence, the following explanation is mainly given about the operations performed at Steps S21A and S22A.

The operations at Steps S21A and S22A are performed when it is determined that a user operation for generating an estimation model is performed (Yes at Step S20).

More particularly, the first processor 123 according to the second embodiment performs machine learning using the training data stored in the memory unit 124 (Step S21A), and generates an estimation model meant for estimating relevant information that is related to the treatment given to the target site (Step S22A). In the second embodiment, the relevant information indicates the temperature of at least either the end effector 9 or the target site.

The estimation model is made of a neural network in which each layer includes one or more nodes. Meanwhile, there is no particular restriction on the type of machine learning. Thus, as long as a plurality of sets of training data generated according to the training data generation method is input for learning in a calculation model that is based on a multilayered neural network; it serves the purpose. As far as the method for machine learning is concerned, for example, a method based on a DNN of a multilayered neural network, such as a CNN (Convolutional Neural Network), can be implemented. Alternatively, as far as the method for machine learning is concerned, a method based on a recurrent neural network (RNN) can be implemented, or a method based on an LSTM, which is obtained by expanding an RNN, can be implemented.

Then, the first processor 123 outputs the generated estimation model to the control device 3 via the first transmission cable CA1. Moreover, the control device 3 stores the estimation model in the memory unit 39.

Control Method

Given below is the explanation of the control method according to the second embodiment.

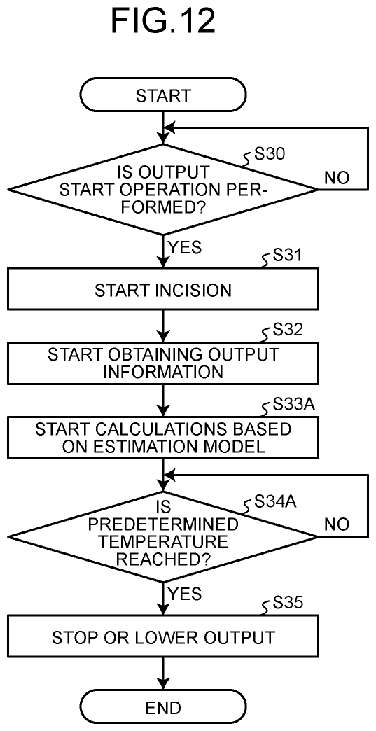

FIG. 12 is a flowchart for explaining the control method.

In the control method implemented by the second processor 38 according to the second embodiment, as illustrated in FIG. 12, with reference to the control method according to the first embodiment (see FIG. 7); Steps S33 and S34 are replaced with Steps S33A and S34A, respectively. Hence, the following explanation is mainly given about the operations performed at Steps S33A and S34A.

The operation at Step S33A is performed after the operation performed at Step S32.

More particularly, at Step S33A, the second processor 38 starts calculations based on the estimation model stored in the memory unit 39.

That is, at Step S33A, the second processor 38 treats the output information (1) to the output information (11) as the input data and performs calculations based on the estimation model; and outputs (estimates) the relevant information related to the treatment given to the target site as output data. In the second embodiment, as explained above, the relevant information indicates the temperature of at least either the end effector 9 or the target site.

After the operation at Step S33A is performed, the second processor 38 refers to the calculation result obtained at Step S33A and constantly monitors whether or not the temperature of at least either the end effector 9 or the target site has reached a predetermined temperature (Step S34A). Until it is determined that the temperature of at least either the end effector 9 or the target site has reached the predetermined temperature, the second processor 38 repeatedly performs the operation at Step S34A. Examples of the predetermined temperature include the temperature that affects the resistance property of the pad provided on the surface of the jaw 6 facing the treatment portion 81, and the temperature that is likely to cause excessive thermal invasion into the surrounding tissues.

More particularly, when the information indicating that the temperature of at least either the end effector 9 or the target site has reached the predetermined temperature is output as the output data at Step S33A, the second processor 38 determines “Yes” at Step S34A. On the other hand, when the information indicating that the temperature of at least either the end effector 9 or the target site has not reached the predetermined temperature is output as the output data at Step S33A, the second processor 38 determines “No” at Step S34A.

When “Yes” is determined at Step S34A, the system control proceeds to Step S35.

According to the second embodiment described above, it becomes possible to achieve the following effects.

In the training data generation method implemented by the first processor 123 according to the second embodiment, the information obtained from the data of photographed images (the temperature information indicating the temperature of at least either the end effector 9 or the target site) is added (labeled) to the output information (1) to the output information (11), and the training data is generated. Moreover, in the estimation model generation method implemented by the first processor 123, machine learning is performed using the training data, and an estimation model is generated. Then, in the control method implemented by the second processor 38, calculations are performed based on the estimation model and it is estimated whether or not the temperature of at least either the end effector 9 or the target site has reached a predetermined temperature, that is, it is estimated whether or not the incision of the target site is complete. If it is determined that the incision of the target site is complete, then the output of the ultrasound energy and the high-frequency energy is stopped. As a result of stopping or lowering the output, the temperature of the target site or the end effector 9 can be prevented from rising to or beyond the predetermined temperature. As a result, no unnecessary damage is caused to the target site or the end effector 9.

Thus, the training data generation method, the control device 3, and the control method according to the second embodiment enable giving appropriate treatment to the target site.

### Other Embodiments

Till now, the description was given about the embodiments of the disclosure. However, the disclosure is not limited by the first and second embodiments described above.

### First Modification Example

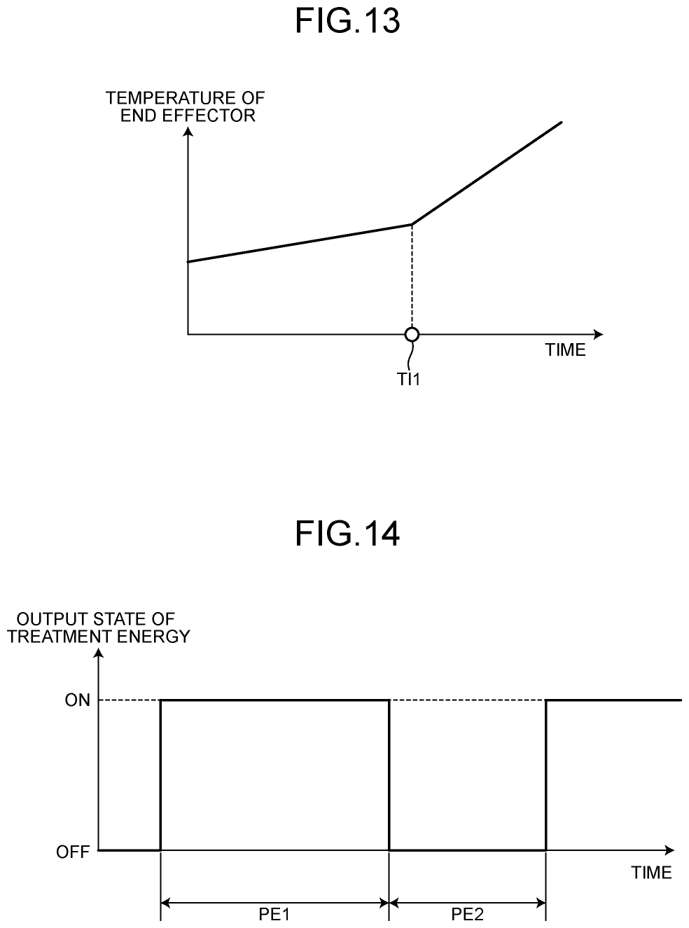

FIG. 13 is a diagram for explaining a first modification example of the first embodiment. More particularly, FIG. 13 is diagram in which the vertical axis represents the temperature of the end effector 9 and the horizontal axis represents the time, and which illustrates the time variation in the temperature after the operation at Step S11 is performed in the training data generation method.

In the training data generation method according to the second embodiment described earlier, at Step S16A, whether or not a predetermined temperature is reached is determined using the treatment completion detection model. However, that is not the only possible case.

Alternatively, for example, the temperature of the end effector 9 is sequentially measured using the photographing device 11A explained in the second embodiment.

Meanwhile, after the target region is incised, the jaw 6 and the treatment portion 81 come in contact with each other. Hence, as illustrated in FIG. 13, after a timing TI1 at which the incision of the target site is complete, there is a sharp increase in the percentage of rise of the temperature of the end effector 9.

Then, based on such variation in the percentage of rise, the first processor 123 recognizes the timing TI1 at which the incision of the target site is complete.

Also in the case in which it is determined that the incision of the target site is complete as explained above in the first modification example, the application of the ultrasound energy and the high-frequency energy is not continued after the completion of the incision of the target site. As a result, no unnecessary damage is caused to the target site or the end effector 9.

Meanwhile, the temperature of the end effector 9 is not limited to be measured using the photographing device 11A, and alternatively can be measured using a temperature sensor such as a thermocouple. In an identical manner, in the second embodiment too, a temperature sensor can be used instead of using the photographing device 11A.

### Second Modification Example

FIG. 14 is a diagram for explaining a second modification example of the first and second embodiments. More particularly, FIG. 14 is a diagram in which the vertical axis represents the output state of the treatment energy, and the horizontal axis represents the time.

In the first and second embodiments described earlier, all sets of output information from the output information (1) to the output information (11) are included as the training data. However, that is not the only possible case. That is, as long as two sets of output information from among the output information (1) to the output information (11) are included, it is also possible to use some other training data.

Moreover, an estimation model explained below according to the second modification example can be used in the first embodiment described earlier.

The estimation model according to the second modification example is generated as a result of performing machine learning using the first training data and the second training data having the following information added (labeled) thereto: at least two sets of information from among the output information (1) to the output information (11); and information about an output period PE1 (see FIG. 14) and a non-application period PE2 (see FIG. 14) as obtained from the data of photographed images (i.e., information indicating that the incision is not yet complete, and information indicating that the incision is complete).

The output period PE1 represents the period of time during which the treatment energy (the ultrasound energy and the high-frequency energy) is applied to the body tissue from the energy treatment tool 2 immediately prior to the present point of time. The non-application period PE2 represents the period of time during which, after completing the immediately preceding application of the treatment energy, the application of the treatment energy is stopped (until the start of application of the treatment energy at the present point of time).

The output period PE1 and the non-application period PE2 represent the information enabling estimation of the temperature of the end effector 9 based on the residual heat present at the time of starting the application of the treatment energy at the present point of time. Depending on the temperature of the end effector 9, the time taken for completing the incision of the target site differs. Hence, the output period PE1 and the non-application period PE2 represent the information enabling estimation of the completion of the incision of the target site.

In an identical manner, the estimation model explained below according to the second modification example can be used in the second embodiment described earlier.

The estimation model according to the second modification example is generated as a result of performing machine learning using the training data having the following information added (labeled) thereto: at least two sets of information from among the output information (1) to the output information (11); and information about the output period PE1 (see FIG. 14) and the non-application period PE2 (see FIG. 14) as obtained from the data of photographed images (i.e., information indicating the temperature of at least either the end effector 9 or the target site).

The estimation model according to the second modification example is made of a neural network in which each layer includes one or more nodes. Meanwhile, there is no particular restriction on the type of machine learning. Thus, as long as a plurality of sets of first training data and second training data (in the case of the second embodiment, a plurality of sets of training data) is prepared and is input for learning in a calculation model that is based on a multilayered neural network; it serves the purpose. As far as the method for machine learning is concerned, for example, a method based on a DNN of a multilayered neural network, such as a CNN, can be implemented. Alternatively, as far as the method for machine learning is concerned, a method based on a recurrent neural network (RNN) can be implemented, or a method based on an LSTM, which is obtained by expanding an RNN, can be implemented.

According to the second modification example explained above, it becomes possible to achieve the following effects in addition to achieving the effects identical to the first and second embodiments described earlier.

In the estimation model according to the second modification example, at least two sets of output information from among the output information (1) to the output information (11) are used along with using the output period PE1 and the non-application period PE2. Hence, the completion of the incision of the target site can be estimated with a higher degree of accuracy, or the temperature of at least either the end effector 9 or the target site can be estimated with a higher degree of accuracy.

Meanwhile, in the estimation model according to the second modification example, although at least two sets of output information from among the output information (1) to the output information (11) are used along with using the output period PE1 and the non-application period PE2, that is not the only possible case. Thus, it is also possible to use the model name of the energy treatment tool 2, the length of the vibration transmission member 8, the model name of the transducer unit 7, and various setting values of the first driving signal and the second driving signal.

### Third Modification Example

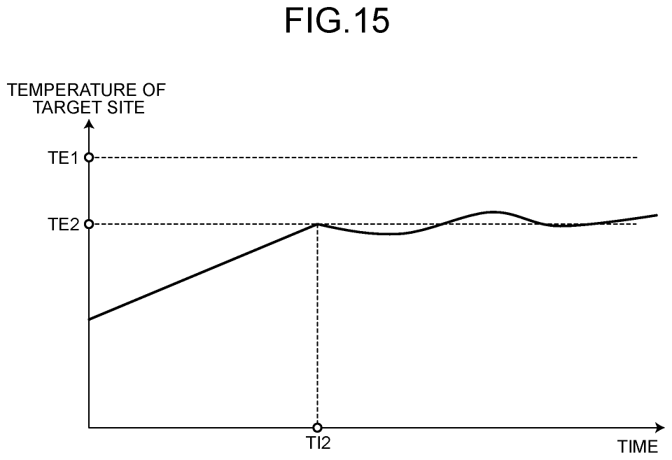

FIG. 15 is a diagram for explaining a third modification example of the second embodiment. More particularly, FIG. 15 is a diagram in which the vertical axis represents the temperature of the target site, and the horizontal axis represents the time. In FIG. 15, a temperature TE1 represents the tolerant temperature of the pad (not illustrated) that is provided on the surface of the jaw 6 facing the treatment portion 81. Moreover, a temperature TE2 represents the temperature at which the protein substance undergoes denaturation. In other words, the temperature TE2 represents the temperature at which the incision of the target site is started.

In the control method according to the second embodiment described earlier, as the predetermined temperature used at Step S34A, the temperature of at least either the end effector 9 or the target site is used because of the reason that it becomes possible to envision the completion of the incision of the target site. However, that is not the only possible case.

Alternatively, for example, as the predetermined temperature, it is possible to use the temperature at which the protein substance undergoes denaturation, that is, the temperature TE2 at which the incision of the target site is started. At that time, if “Yes” is determined at Step S34A, then the second processor 38 controls the operations of the first power source 31 and the second power source 34 so as to either lower the output of the ultrasound energy and the high-frequency energy or cause intermittent output of the ultrasound energy and the high-frequency energy; and, as illustrated in FIG. 15, performs control to maintain the temperature of the target site in the vicinity of the temperature TE2.

According to the third modification example explained above, it becomes possible to achieve the following effects in addition to achieving the effects identical to the second embodiment described earlier.

In the control method according to the third modification example, since the control is performed to maintain the temperature of the target site in the vicinity of the temperature TE2, it becomes possible to avoid a situation in which the heat of the target site affects the resistance of the pad that is provided on the surface of the jaw 6 facing the treatment portion 81.

### Fourth Modification Example

In the first embodiment described earlier, it is possible to use a pressure-resistance estimation model explained below according to a fourth modification example.

The pressure-resistance estimation model is generated as a result of performing machine learning using training data in which at least two sets of output information from among the output information (1) to the output information (11) are associated to the sealing pressure-resistance of a blood vessel, which represents the target site, as measured at the time of detection of the two sets of output information.

The pressure-resistance estimation model is made of a neural network including one or more nodes. Meanwhile, there is no particular restriction on the type of machine learning. Thus, as long as a plurality of sets of training data, in which at least two sets of output information from among the output information (1) to the output information (11) are associated to the sealing pressure-resistance of a blood vessel as measured at the time of detection of the two sets of output information, is prepared and is input for learning in a calculation model that is based on a multilayered neural network; it serves the purpose. As far as the method for machine learning is concerned, for example, a method based on a DNN of a multilayered neural network, such as a CNN, can be implemented. Alternatively, as far as the method for machine learning is concerned, a method based on a recurrent neural network (RNN) can be implemented, or a method based on an LSTM, which is obtained by expanding an RNN, can be implemented.

Then, at the time of implementing the control method, the second processor 38 treats at least two sets of output information, from among the output information (1) to the output information (11), as the input data; and performs calculations based on the pressure-resistance estimation model so as to output (estimate) the sealing pressure-resistance of the blood vessel as the output data. Moreover, immediately prior to the estimated timing of completion of the incision of the blood vessel as estimated by performing calculations based on the estimation model, if it is determined that the sealing pressure-resistance of the blood vessel is lower than a specific sealing pressure-resistance, then the second processor 38 performs control in the following manner.

That is, the second processor 38 controls the operations of the first power source 31 and the second power source 34 so as to lower the output of the ultrasound energy and to increase the output of the high-frequency energy, and sets the sealing pressure-resistance of the blood vessel at a sufficiently high level.

According to the fourth modification example explained above, it becomes possible to achieve the following effects in addition to achieving the effects identical to the first and second embodiments described earlier.

In the control method according to the fourth modification example, immediately prior to the timing of completion of the incision of the blood vessel, when it is determined that the sealing pressure-resistance of the blood vessel is lower than a specific sealing pressure-resistance, the abovementioned control is performed so that it becomes possible to complete the treatment with the sealing pressure-resistance maintained at a sufficiently high level.

### Fifth Modification Example

The second processor 38 can implement a control method in, for example, the manner explained below by using the estimation model according to the first embodiment as well as using the estimation model according to the second embodiment.

When the estimated temperature of at least either the end effector 9 or the target site, which is estimated by performing calculations based on the estimation model according to the second embodiment, reaches a predetermined temperature; the second processor 38 controls the operations of the first power source 31 and the second power source 34, and lowers the output of the ultrasound energy and the high-frequency energy. Then, if the incision of the target site is estimated by performing calculations based on the estimation model according to the first embodiment, the second processor 38 stops the operations of the first power source 31 and the second power source 34.

Meanwhile, in the first and second embodiments described earlier, it is possible to use at least one front-end estimation model from among a first front-end estimation model to a ninth front-end estimation model explained below according to a fifth modification example.

The first front-end estimation model is generated as a result of performing machine learning using training data in which at least two sets of output information from among the output information (1) to the output information (11) are associated to the type of the target site. Examples of the type of the target site include a blood vessel, the liver, the cervix, a membrane tissue, a parenchyma organ, a muscle tissue, and a rigid tissue.

The first front-end estimation model is made of a neural network in which each layer includes one or more nodes. Meanwhile, there is no particular restriction on the type of machine learning. Thus, as long as a plurality of sets of training data, in which at least two sets of output information from among the output information (1) to the output information (11) are associated to the type of the target site, is prepared and is input for learning in a calculation model that is based on a multilayered neural network; it serves the purpose. As far as the method for machine learning is concerned, for example, a method based on a DNN of a multilayered neural network, such as a CNN, can be implemented. Alternatively, as far as the method for machine learning is concerned, a method based on a recurrent neural network (RNN) can be implemented, or a method based on an LSTM, which is obtained by expanding an RNN, can be implemented.

Then, at the time of implementing the control method, the second processor 38 treats at least two sets of output information, from among the output information (1) to the output information (11), as the input data and performs calculations based on the first front-end estimation model; and outputs (estimates) the type of the target site, which is grasped between the jaw 6 and the treatment portion 81, as the output data. Herein, each estimation model is generated according to a type of the target site. Thus, using the estimation model corresponding to the estimated type of the target site, the second processor 38 estimates the completion of the incision of the target site or estimates the temperature of at least either the end effector 9 or the target site.

The second front-end estimation model is generated as a result of performing machine learning using training data in which at least two sets of output information from among the output information (1) to the output information (11) are associated to the grasping length. The grasping length implies the proportion of the length of the grasped target site, which is grasped between the jaw 6 and the treatment portion 81, with respect to the total length of at least either the jaw 6 or the treatment portion 81.

The second front-end estimation model is made of a neural network in which each layer includes one or more nodes. Meanwhile, there is no particular restriction on the type of machine learning. Thus, as long as a plurality of sets of training data, in which at least two sets of output information from among the output information (1) to the output information (11) are associated to the grasping length, is prepared and is input for learning in a calculation model that is based on a multilayered neural network; it serves the purpose. As far as the method for machine learning is concerned, for example, a method based on a DNN of a multilayered neural network, such as a CNN, can be implemented. Alternatively, as far as the method for machine learning is concerned, a method based on a recurrent neural network (RNN) can be implemented, or a method based on an LSTM, which is obtained by expanding an RNN, can be implemented.

Then, at the time of implementing the control method, the second processor 38 treats at least two sets of output information, from among the output information (1) to the output information (11), as the input data and performs calculations based on the second front-end estimation model; and outputs (estimates) the grasping length as the output data. Herein, each estimation model is generated according to a grasping length. Thus, using the estimation model corresponding to the estimated grasping length, the second processor 38 estimates the completion of the incision of the target site or estimates the temperature of at least either the end effector 9 or the target site.

The third front-end estimation model is generated as a result of performing machine learning using training data in which at least two sets of output information from among the output information (1) to the output information (11) are associated to the hardness of the body tissue.

The third front-end estimation model is made of a neural network in which each layer includes one or more nodes. Meanwhile, there is no particular restriction on the type of machine learning. Thus, as long as a plurality of sets of training data, in which at least two sets of output information from among the output information (1) to the output information (11) are associated to the hardness of the body tissue, is prepared and is input for learning in a calculation model that is based on a multilayered neural network; it serves the purpose. As far as the method for machine learning is concerned, for example, a method based on a DNN of a multilayered neural network, such as a CNN, can be implemented. Alternatively, as far as the method for machine learning is concerned, a method based on a recurrent neural network (RNN) can be implemented, or a method based on an LSTM, which is obtained by expanding an RNN, can be implemented.

Then, at the time of implementing the control method, the second processor 38 treats at least two sets of output information, from among the output information (1) to the output information (11), as the input data and performs calculations based on the third front-end estimation model; and outputs (estimates) the hardness of the target site, which is grasped between the jaw 6 and the treatment portion 81, as the output data. Herein, each estimation model is generated according to a hardness. Thus, using the estimation model corresponding to the estimated hardness of the target site, the second processor 38 estimates the completion of the incision of the target site or estimates the temperature of at least either the end effector 9 or the target site.

The fourth front-end estimation model is generated as a result of performing machine learning using training data in which at least two sets of output information from among the output information (1) to the output information (11) are associated to the contamination condition of at least either the jaw 6 or the treatment portion 81.

The fourth front-end estimation model is made of a neural network in which each layer includes one or more nodes. Meanwhile, there is no particular restriction on the type of machine learning. Thus, as long as a plurality of sets of training data, in which at least two sets of output information from among the output information (1) to the output information (11) are associated to the contamination condition of at least either the jaw 6 or the treatment portion 81, is prepared and is input for learning in a calculation model that is based on a multilayered neural network; it serves the purpose. As far as the method for machine learning is concerned, for example, a method based on a DNN of a multilayered neural network, such as a CNN, can be implemented. Alternatively, as far as the method for machine learning is concerned, a method based on a recurrent neural network (RNN) can be implemented, or a method based on an LSTM, which is obtained by expanding an RNN, can be implemented.

Then, at the time of implementing the control method, the second processor 38 treats at least two sets of output information, from among the output information (1) to the output information (11), as the input data and performs calculations based on the fourth front-end estimation model; and outputs (estimates) the contamination condition of at least the jaw 6 or the treatment portion 81. Herein, each estimation model is generated according to a contamination condition of at least the jaw 6 or the treatment portion 81. Thus, using the estimation model corresponding to the estimated contamination condition of at least either the jaw 6 or the treatment portion 81, the second processor 38 estimates the completion of the incision of the target site or estimates the temperature of at least either the end effector 9 or the target site.

The fifth front-end estimation model is generated as a result of performing machine learning using training data in which at least two sets of output information from among the output information (1) to the output information (11) are associated to the abrasion condition of at least either the jaw 6 or the treatment portion 81.

The fifth front-end estimation model is made of a neural network in which each layer includes one or more nodes. Meanwhile, there is no particular restriction on the type of machine learning. Thus, as long as a plurality of sets of training data, in which at least two sets of output information from among the output information (1) to the output information (11) are associated to the abrasion condition of at least either the jaw 6 or the treatment portion 81, is prepared and is input for learning in a calculation model that is based on a multilayered neural network; it serves the purpose. As far as the method for machine learning is concerned, for example, a method based on a DNN of a multilayered neural network, such as a CNN, can be implemented. Alternatively, as far as the method for machine learning is concerned, a method based on a recurrent neural network (RNN) can be implemented, or a method based on an LSTM, which is obtained by expanding an RNN, can be implemented.

Then, at the time of implementing the control method, the second processor 38 treats at least two sets of output information, from among the output information (1) to the output information (11), as the input data and performs calculations based on the fifth front-end estimation model; and outputs (estimates) the abrasion condition of at least the jaw 6 or the treatment portion 81. Herein, each estimation model is generated according to an abrasion condition of at least the jaw 6 or the treatment portion 81 as the output data. Thus, using the estimation model corresponding to the estimated abrasion condition of at least either the jaw 6 or the treatment portion 81, the second processor 38 estimates the completion of the incision of the target site or estimates the temperature of at least either the end effector 9 or the target site.

The sixth front-end estimation model is generated as a result of performing machine learning using training data in which at least two sets of output information from among the output information (1) to the output information (11) are associated to the layered structure of the body tissue. Examples of the layered structure include a single-membrane structure or a multilayered structure.

The sixth front-end estimation model is made of a neural network in which each layer includes one or more nodes. Meanwhile, there is no particular restriction on the type of machine learning. Thus, as long as a plurality of sets of training data, in which at least two sets of output information from among the output information (1) to the output information (11) are associated to the layered structure of the body tissue, is prepared and is input for learning in a calculation model that is based on a multilayered neural network; it serves the purpose. As far as the method for machine learning is concerned, for example, a method based on a DNN of a multilayered neural network, such as a CNN, can be implemented. Alternatively, as far as the method for machine learning is concerned, a method based on a recurrent neural network (RNN) can be implemented, or a method based on an LSTM, which is obtained by expanding an RNN, can be implemented.

Then, at the time of implementing the control method, the second processor 38 treats at least two sets of output information, from among the output information (1) to the output information (11), as the input data and performs calculations based on the sixth front-end estimation model; and outputs (estimates) the layered structure of the target site, which is grasped between the jaw 6 and the treatment portion 81, as the output data. Herein, each estimation model is generated according to a layered structure. Thus, using the estimation model corresponding to the estimated layered structure of the target site, the second processor 38 estimates the completion of the incision of the target site or estimates the temperature of at least either the end effector 9 or the target site.

The seventh front-end estimation model is generated as a result of performing machine learning using training data in which at least two sets of output information from among the output information (1) to the output information (11) are associated to the component of the body tissue. Examples of the component include collagen and the proportion of fat.

The seventh front-end estimation model is made of a neural network in which each layer includes one or more nodes. Meanwhile, there is no particular restriction on the type of machine learning. Thus, as long as a plurality of sets of training data, in which at least two sets of output information from among the output information (1) to the output information (11) are associated to the component of the body tissue, is prepared and is input for learning in a calculation model that is based on a multilayered neural network; it serves the purpose. As far as the method for machine learning is concerned, for example, a method based on a DNN of a multilayered neural network, such as a CNN, can be implemented. Alternatively, as far as the method for machine learning is concerned, a method based on a recurrent neural network (RNN) can be implemented, or a method based on an LSTM, which is obtained by expanding an RNN, can be implemented.

Then, at the time of implementing the control method, the second processor 38 treats at least two sets of output information, from among the output information (1) to the output information (11), as the input data and performs calculations based on the seventh front-end estimation model; and outputs (estimates) the component of the target site, which is grasped between the jaw 6 and the treatment portion 81, as the output data. Herein, each estimation model is generated according to a component. Thus, using the estimation model corresponding to the estimated component of the target site, the second processor 38 estimates the completion of the incision of the target site or estimates the temperature of at least either the end effector 9 or the target site.

The eighth front-end estimation model is generated as a result of performing machine learning using training data in which at least two sets of output information from among the output information (1) to the output information (11) are associated to the grasping force. In the state in which the target site is not grasped between the jaw 6 and the treatment portion 81, the grasping force is treated to be at 0%. In the state in which the operation knob 41 is operated to the maximum extent possible, the grasping force is treated to be at 100%. Thus, the grasping force is expressed using a value between 0% and 100%.

The eighth front-end estimation model is made of a neural network in which each layer includes one or more nodes. Meanwhile, there is no particular restriction on the type of machine learning. Thus, as long as a plurality of sets of training data, in which at least two sets of output information from among the output information (1) to the output information (11) are associated to the grasping force, is prepared and is input for learning in a calculation model that is based on a multilayered neural network; it serves the purpose. As far as the method for machine learning is concerned, for example, a method based on a DNN of a multilayered neural network, such as a CNN, can be implemented. Alternatively, as far as the method for machine learning is concerned, a method based on a recurrent neural network (RNN) can be implemented, or a method based on an LSTM, which is obtained by expanding an RNN, can be implemented.

Then, at the time of implementing the control method, the second processor 38 treats at least two sets of output information, from among the output information (1) to the output information (11), as the input data and performs calculations based on the eighth front-end estimation model; and outputs (estimates) the grasping force as the output data. Herein, each estimation model is generated according to a grasping force. Thus, using the estimation model corresponding to the estimated grasping force, the second processor 38 estimates the completion of the incision of the target site or estimates the temperature of at least either the end effector 9 or the target site.

The ninth front-end estimation model is generated as a result of performing machine learning using training data in which at least two sets of output information from among the output information (1) to the output information (11) are associated to the environment at the front end of the end effector 9. Examples of that environment include the laparoscopy environment, the laparotomy environment, the normal saline solution environment, and the inside-the-blood environment.

The ninth front-end estimation model is made of a neural network in which each layer includes one or more nodes. Meanwhile, there is no particular restriction on the type of machine learning. Thus, as long as a plurality of sets of training data, in which at least two sets of output information from among the output information (1) to the output information (11) are associated to the environment at the front end of the end effector 9, is prepared and is input for learning in a calculation model that is based on a multilayered neural network; it serves the purpose. As far as the method for machine learning is concerned, for example, a method based on a DNN of a multilayered neural network, such as a CNN, can be implemented. Alternatively, as far as the method for machine learning is concerned, a method based on a recurrent neural network (RNN) can be implemented, or a method based on an LSTM, which is obtained by expanding an RNN, can be implemented.

Then, at the time of implementing the control method, the second processor 38 treats at least two sets of output information, from among the output information (1) to the output information (11), as the input data and performs calculations based on the ninth front-end estimation model; and outputs (estimates) the environment at the front end of the end effector 9 as the output data. Herein, each estimation model is generated according to an environment. Thus, using the estimation model corresponding to the estimated environment, the second processor 38 estimates the completion of the incision of the target site or estimates the temperature of at least either the end effector 9 or the target site.

According to the fifth modification example explained above, it becomes possible to achieve the following effects in addition to achieving the effects identical to the first and second embodiments described earlier.

In the fifth modification example, since at least one front-end estimation model from among the first front-end estimation model to the ninth front-end estimation model is used, the completion of the incision of the target site can be estimated with a higher degree of accuracy, or the temperature of at least either the end effector 9 or the target site can be estimated with a higher degree of accuracy.

Meanwhile, it is also possible to combine two or more front-end estimation models from among the first front-end estimation model to the ninth front-end estimation model. Moreover, in the first front-end estimation model to the ninth front-end estimation model explained above, although at least two sets of output information from among the output information (1) to the output information (11) are used, that is not the only possible case. Alternatively, it is possible to use photographed images that are photographed using an endoscope, or to use the output values detected by sensors installed in the energy treatment tool 2.

### Sixth Modification Example

In the first and second embodiments described above, the ultrasound energy and the high-frequency energy is used as the treatment energy to be applied to the target site. However, that is not the only possible case. Alternatively, it is possible to use only the ultrasound energy.

The training data generation method, the control device, and the control method according to the disclosure enable giving appropriate treatment to the body tissue.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A training data generation method implemented by a processor of a training data generation device, the training data generation method comprising:
obtaining output information related to an electrical characteristic value in an energy treatment tool when ultrasound energy is being applied from the energy treatment tool to a body tissue;
obtaining photography data that contains a photograph taken of a state in which the ultrasound energy is being applied to the body tissue;
obtaining a label from the photography data; and
adding the label to the output information to generate the training data.

2. The training data generation method according to claim 1, wherein the electrical characteristic value includes at least two of:
an electric current value to be supplied to an ultrasound transducer which generates the ultrasound energy in the energy treatment tool,
a voltage value to be supplied to the ultrasound transducer,
an electric power value to be supplied to the ultrasound transducer,
frequency of electric current or frequency of voltage to be supplied to the ultrasound transducer,
an ultrasound impedance value calculated from the electric current value and the voltage value, and
elapsed time since start of application of the ultrasound energy to the body tissue.

3. The training data generation method according to claim 1, wherein the photography data is taken using thermography and contains temperature information indicating temperature of an end effector of the energy treatment tool.

4. The training data generation method according to claim 1, wherein the photography data is taken using thermography and contains temperature information indicating temperature of the body tissue.

5. The training data generation method according to claim 1, wherein the label indicates whether the body tissue is incised as a result of application of the ultrasound energy.

6. The training data generation method according to claim 1, wherein
the obtaining of the output information includes obtaining the output information related to the electrical characteristic value in the energy treatment tool when high-frequency energy is being applied to the body tissue along with application of the ultrasound energy, and
the obtaining of the photography data includes obtaining the photography data that contains a photograph taken of a state in which the high-frequency energy is being applied to the body tissue along with the application of the ultrasound energy.

7. The training data generation method according to claim 6, wherein the electrical characteristic value includes at least one of:
an electric current value to be supplied to a pair of electrodes that generate the high-frequency energy in the energy treatment tool,
a voltage value to be supplied to the pair of electrodes,
an electric power value to be supplied to the pair of electrodes,
a phase difference between electric current and voltage to be supplied to the pair of electrodes,
an impedance value of the body tissue as calculated from the electric current value and the voltage value,
a resistance of the body tissue as obtained by multiplying the phase difference to the impedance value, and
elapsed time since start of application of the high-frequency energy to the body tissue.

8. A control device comprising a processor, the processor being configured to:
obtain output information related to an electrical characteristic value in an energy treatment tool when ultrasound energy is being applied from the energy treatment tool to a body tissue;
input the output information to an estimation model generated as a result of performing machine learning; and
obtain relevant information related to treatment of the body tissue from the estimation model.

9. The control device according to claim 8, wherein the electrical characteristic value includes at least two of:
an electric current value to be supplied to an ultrasound transducer which generates the ultrasound energy in the energy treatment tool,
a voltage value to be supplied to the ultrasound transducer,
an electric power value to be supplied to the ultrasound transducer,
frequency of electric current or frequency of voltage to be supplied to the ultrasound transducer,
an ultrasound impedance value calculated from the electric current value and the voltage value, and
elapsed time since start of application of the ultrasound energy to the body tissue.

10. The control device according to claim 8, wherein the relevant information indicates whether an incision of the body tissue is complete.

11. The control device according to claim 10, further comprising a power source configured to output a driving signal to an ultrasound transducer in the energy treatment tool for causing generation of the ultrasound energy,
wherein, when the relevant information indicates that the incision of the body tissue is complete, the processor is configured to control operation of the power source to stop or lower output of the ultrasound energy.

12. The control device according to claim 8, wherein the relevant information indicates temperature of at least one of an end effector in the energy treatment tool and the body tissue.

13. The control device according to claim 12, further comprising a power source configured to output a driving signal to an ultrasound transducer in the energy treatment tool for causing generation of the ultrasound energy,
wherein, when the temperature of the at least one of the end effector and the body tissue reaches a predetermined temperature, the processor is configured to control operation of the power source to stop or lower output of the ultrasound energy.

14. The control device according to claim 12, further comprising a power source configured to output a driving signal to an ultrasound transducer in the energy treatment tool for causing generation of the ultrasound energy,
wherein, when the temperature of the at least one of the end effector and the body tissue reaches a predetermined temperature, the processor is configured to control operation of the power source to lower output of the ultrasound energy or cause intermittent output of the ultrasound energy.

15. The control device according to claim 8, wherein the estimation model is generated using deep learning.

16. The control device according to claim 8, wherein the output information is output information related to an electrical characteristic value in the energy treatment tool when high-frequency energy is being applied to the body tissue along with application of the ultrasound energy.

17. The control device according to claim 16, wherein the electrical characteristic value includes at least one of:
an electric current value to be supplied to a pair of electrodes that generate the high-frequency energy in the energy treatment tool,
a voltage value to be supplied to the pair of electrodes,
an electric power value to be supplied to the pair of electrodes,
a phase difference between electric current and voltage to be supplied to the pair of electrodes,
an impedance value of the body tissue as calculated from the electric current value and the voltage value,
a resistance of the body tissue as obtained by multiplying the phase difference to the impedance value, and
elapsed time since start of application of the high-frequency energy to the body tissue.

18. The control device according to claim 8, wherein the output information contains:
an output period during which the ultrasound energy was being applied to the body tissue from the energy treatment tool immediately prior to present point of time, and
a non-output period during which, after completion of immediately preceding application of the ultrasound energy, the application is being stopped.

19. A control method implemented by a processor of a control device, the control method comprising:
obtaining output information related to an electrical characteristic value in an energy treatment tool when ultrasound energy is being applied from the energy treatment tool to a body tissue;
inputting the output information to an estimation model generated as a result of performing machine learning; and
obtaining relevant information related to treatment of the body tissue from the estimation model.

20. The training data generation method according to claim 1, wherein the training data is to be used in machine learning performed at time of generating an estimation model for estimating relevant information, the relevant information is related to treatment of the body tissue.
